(19) United States
(12) Patent Application Publication
(10) Pub. No.: US 2020/0055045 A1
(43) Pub. Date: Feb. 20, 2020

(54) NANOFLUIDIC PLATFORM

(71) Applicant: The Royal Institution for the Advancement of Leaming/McGill University, Montreal (CA)

(72) Inventors: Sabrina R. LESLIE, Montreal (CA); Gilead P. HENKIN, London (GB); Daniel J. BERARD, Montreal (CA)

(21) Appl. No.: 16/347,211

(22) PCT Filed: Apr. 20, 2017
(86) PCT No.: PCT/IB2017/000555

§ 371 Date: May 3, 2019

Related U.S. Application Data
(60) Provisional application No. 62/416,935, filed on Nov. 3, 2016.

(51) Int. Cl. B01L 3/00; G01N 15/14; G01N 33/53

(52) U.S. Cl. CPC B01L 3/502715; G01N 21/6428; G01N 33/5308; G01N 15/1436

(57) ABSTRACT

Provided herein are devices for manipulating and visualizing molecular interactions in customized nanoscale spaces.

### RELATED APPLICATION

This application claims the benefit under 35 U.S.C. § 119(e) of U.S. provisional application Ser. No. 62/416,935, filed Nov. 3, 2016, the disclosure of which is incorporated by reference herein in its entirety.

### FIELD

Provided herein are devices for manipulating and visualizing molecular interactions in customized nanoscale spaces.

### BACKGROUND

As fluorescence microscopy has advanced to the point of single-molecule resolution, there is growing interest in visualizing and quantitatively understanding biochemical mechanisms at the nanoscale. Single-molecule microscopy of DNA molecules undergoing dynamic processes and interactions inside nanoscale volumes, such as DNA condensation in the presence of proteins or crowding agents, can provide important mechanistic insights into physiological processes occurring under confinement, such as in the dense nucleus of a cell.

Further, recent studies show that in vitro nanoconfinement of enzymatic processes can enhance reactivity.¹ Single-DNA manipulation and visualization techniques represent the cutting edge of not only discovering biophysical mechanisms at the nanoscale, but also of developing third-generation DNA sequencing and DNA mapping technologies, which seek to load and analyze complex genomic material within nanoscale spaces.²⁻⁶

Despite a flurry of technology development, existing single-molecule approaches to probing biomolecular processes face challenges in simultaneously achieving reproducible throughput and sensitivity, as well as temporal control and resolution of dynamics and kinetics, over a wide range of reagent and imaging conditions. Retaining structural integrity of complex genomic samples while overcoming several orders of magnitude of applied confinement during sample-loading presents an additional challenge.^{7,8}

Prior single-molecule microscopy of molecular interactions has typically used surface immobilized or tethered DNA molecules for convenient data collection and analysis. While Total Internal Reflection Fluorescence (TIRF) microscopy has enabled exquisite visualization of interactions between surface-immobilized and freely diffusing molecules, providing important biophysical insights,⁹ these measurements can be limited by surface and tethering effects, as well as low accessible concentrations and short per-molecule trajectories of the diffusing species.

Furthermore, a range of micro- and nanofluidic fabrication approaches have been developed to achieve sub-persistence-length confinement of DNA and other polymers. By extending DNA polymers in nanochannels, genomic features may be mapped to positions along the extended DNA length^{10,11}, of interest to advancing sequencing and mapping applications. Visualizing interactions between extended DNA and biomolecules allows sequence-specific processes to be probed and understood, such as targeted labeling,¹² binding,¹³ or cleavage.¹⁴ Existing nanofluidic approaches typically load DNA molecules into nanochannels from the side using electrophoresis for in-situ visualization.^{11,15-18}

PDMS-based microfluidics have provided complementary approaches for important proof-of-principle studies, ranging from drug discovery to biomarker detection to genomic analysis platforms.¹⁹ PDMS is often used for rapid prototyping and production of single experiment devices because it is a porous material and risks contamination with re-use. Work by Zhang et al, in particular, showed one method for interfacing reagents with confined DNA in a fluidic PDMS device.²⁰⁻²² Their fabrication methods allowed average nanochannel cross-sectional diameter down to 200 nm.

### SUMMARY

Presented herein is a dynamically adjustable nanofluidic platform for formatting the conformations of and visualizing the interaction kinetics between biomolecules in solution, offering new time resolution and control of the reaction processes. This platform extends Convex Lens-induced Confinement (CLiC), a method for imaging molecules under confinement, by introducing a system for in-situ modification of the chemical environment; this system uses a deep microchannel to diffusively exchange reagents within the nanoscale imaging region, whose height is fixed by a nanopost array.

To illustrate, salt-induced, surfactant-induced, and enzyme-induced reactions between small-molecule reagents and DNA molecules were visualized and manipulated, where the conformations of the DNA molecules are formatted by the imposed nanoscale confinement. In response to dynamically modifying the local salt concentration, two salt-induced transitions in DNA molecules were observed which occur on separate timescales: a rapid change in polymer extension due to modified local ionic screening; and a gradual change in polymer brightness, reflecting release of intercalated YOYO-1 dye. The time-resolved measurements provide new insights into the influence of YOYO-1 dye on polymer stiffness.

In response to introducing cationic surfactants in solution, single-molecule compaction trajectories of DNA polymers were temporally resolved, guided by the confining nanogroove environment; this is in contrast to the uncontrolled collapse which would occur in free solution under similar conditions.

In the presence of restriction enzymes, the cleavage of multiple DNA sites under adjustable nanoscale confinement were directly visualized. By using nanofabricated, non-absorbing, low-background glass walls to confine biomolecules, the disclosed nanofluidic platform facilitates quantitative exploration of physiologically and biotechnologically relevant processes at the nanoscale. This device provides new kinetic information about dynamic chemical processes at the single-molecule level, using advancements in the CLiC design including a microchannel-based diffuser and post-array-based dialysis slit.

According to one aspect, nanofluidic devices are provided that include a flow-cell formed between flow-cell surfaces, wherein at least one of the flow-cell surfaces includes an array of posts, and wherein the flow-cell has variable height. The post array ensures an even confinement around the region of contact of the post array and the flow-cell surfaces, such that the contact of the post array and the flow-cell surfaces creates a minimum chamber height.

In some embodiments, one or more of the flow-cell surfaces include embedded micro- and/or nano-topographies. In some embodiments, one or more of the flow-cell surfaces are formed by a coverslip. In some embodiments, one or more of the flow-cell surfaces include glass and/or silicon surfaces or are formed of glass and/or silicon.

In some embodiments, one or more of the flow-cell surfaces includes a hexagonal array of post extrusions. In some embodiments, the posts forming the array are 30-m-spaced posts. In some embodiments, the posts forming the array are 5-100 nm tall. In some embodiments, the post array forms a nanoslit.

In some embodiments, one of the flow-cell surfaces includes linear embedded nanogrooves. In some embodiments, the nanogrooves are about 40-50 nm deep, about 50-nm wide and about 500-μm long.

In some embodiments, walls of the flow-cell are coated with a surface-passivation agent, optionally polyvinyl pyrrolidone (PVP), and/or a heterobifunctional linker, such as (aminopropyl)triethoxysilane (APTES).

In some embodiments, the flow-cell includes a floor, and the floor of the flow-cell includes a microchannel. In some embodiments, the microchannel is about 30-μm-deep and about 200-μm-wide. In some embodiments, the microchannel encircles the nanogroove array and imaging region.

In some embodiments, the variable height of the flow-cell is controlled by an external mechanism.

In some embodiments, the flow-cell surfaces are substantially planar.

In some embodiments, the flow-cell can be disassembled.

In another aspect, methods for analyzing the interaction of biomolecules are provided. The methods include loading one or more biomolecules and optionally one or more reagents into the flow-cell as described herein, and applying pressure on a flow-cell surface, such that the flow-cell surface contacts the post array. In some embodiments, the pressure is applied by contacting the upper flow-cell surface with a Convex Lens-induced Confinement (CLiC)-lens. In some embodiments, the methods also include obtaining images of the one or more biomolecules.

In some embodiments, the methods also include adding a reagent or coating to the flow-cell that enables deposition, attachment or tethering of biomolecules to one or more surfaces of the flow-cell. In some embodiments, the reagent or coating that enables deposition, attachment or tethering of biomolecules is a heterobifunctional linker, such as (aminopropyl)triethoxysilane (APTES).

Other aspects, embodiments and features of the invention will become apparent from the following detailed description when considered in conjunction with the accompanying drawing(s). The accompanying figures are schematic and are not intended to be drawn to scale. In the figures, each identical, or substantially similar component that is illustrated in various figures may be represented by a single numeral or notation (though not always). For purposes of clarity, not every component is labeled in every figure. Nor is every component of each embodiment shown where illustration is not necessary to allow those of ordinary skill in the art to understand the invention. All patent applications and patents incorporated herein by reference are incorporated by reference in their entirety. In case of conflict, the present specification, including definitions, will control.

### BRIEF DESCRIPTION OF THE DRAWINGS

FIGS. 1a-1f: Nanofluidic device (not to scale unless otherwise noted). FIG. 1a) Schematic of DNA confinement and reagent introduction process. The top surface is deformed downward. When the slit height approaches the 50-nm double-stranded-DNA persistence length, DNA enter and extend along open-face 50-nm nanogrooves. Reagents can be introduced from the surrounding microchannel. FIG. 1b) Schematic of micro- and nanofluidic flow cell. An embedded microchannel in the bottom surface delivers reagents to the central nanogroove array, where DNA is extended and trapped, from inlet holes in the top coverslip. Reagents can diffuse through the post array on the top coverslip, which also provides stability during flow. Coverslips are held together by 10-μm adhesive with a laser-cut flow channel. FIG. 1c) Side-view schematic of device. The CLiC-lens is used to mechanically deform the flow cell and is not used in any optical path. FIG. 1d) Optical microscope image of the central nanochannel array and encircling microchannel. FIG. 1e) SEM image of nanochannel array (inset: nanochannel cross-section). FIG. 1f) Fluorescent intensity of the central CLiC region after introduction of Cy3 fluorescent dye, excited by a 532-nm laser (82×82 microns field of view). A lag on the order of 10 seconds corresponds to the time it takes for the dye to diffuse into the field of view after insertion.

FIGS. 2a and 2b: DNA confinement in the device. FIG. 2a) Schematic of DNA polymers (linear molecules) confined to nanochannels in a buffer containing a fluorescent dye (dots). FIG. 2b) False-color fluorescent image of confined YOYO-1-stained λ-phage DNA (light linear molecules) in buffer containing Cy5 dye, used to image the extruded posts (dark circles). The sample is illuminated with 488- and 647-nm excitation lasers, for YOYO-1 and Cy5 respectively; this image superimposes spectrally separate fluorescence images.

FIGS. 3a-3d: DNA extension and intensity in response to increasing ionic strength. FIG. 3a) Kymogram of a confined YOYO-1-stained λ DNA molecule in response to buffer exchange from 0 to 171 mM added NaCl. The dashed line indicates the time of buffer injection. FIG. 3b) Schematic of proposed mechanism of DNA extension and intensity changes under increased ionic strength. The negative charges along the backbone are screened, decreasing persistence length. At the same time, the intercalated and electrostatically-coupled YOYO-1 molecules dissociate. FIG. 3c) Fitting procedure for extracting DNA length and fluorescent intensity from experimental data. FIG. 3d) Normalized length and intensity of 5-10 DNA molecules (in each instance from a single experiment) for a control with no added salt, as well as three experiments with salt concentrations increasing from 0 to 34, 171, and 855 mM NaCl. The shaded regions indicate the standard deviation. I₀ and L₀ are measured per DNA molecule just before introduction of a new buffer solution. Introducing the 855 mM NaCl buffer causes the DNA intensity to drop to background within 40 seconds, after which the trace is not shown.

FIGS. 4a-4c: DNA extension and fluorescence intensity in response to salt-induced conformational changes. FIG. 4a) DNA are confined in nanogrooves in the absence of NaCl. FIG. 4b) 171 mM NaCl is introduced via the microchannel. FIG. 4c) The NaCl-containing buffer is again replaced with an NaCl-free buffer introduced through the microchannel. Kymographs are corrected for center of mass. Length and intensity measures (following procedures in FIG. 3c) represent averages of 5-10 molecules, with the shaded region indicating standard deviation.

FIG. 5a-5c: λ-phage DNA compaction in response to CTAC. FIG. 5a) Images of Cy3-labeled DNA in nanochannels after introduction of CTAC. Compaction of DNA proceeds with the diffusing front. FIG. 5b) Mean fractional extension of 7 DNA molecules undergoing compaction in one experiment. Trajectories have been aligned to maximize correlation, where to indicates initiation of compaction. Shaded region indicates standard deviation. Red dots indicate the trajectory of a single representative molecule. FIG. 5c) Kymogram of the representative DNA molecule through the compaction transition. Compaction nucleates at the end of the DNA oriented towards the nearest microchannel.

FIGS. 6a-6c: Kymographs of YOYO-1 stained λ-phage DNA being digested by the restriction enzymes FIG. 6a) SacI (with 100-nm posts), FIG. 6b) SmaI (with 40-nm posts), and FIG. 6c) NcoI (with 5-nm posts) after introduction of buffer containing 1 mM MgCl₂. These kymographs feature time increasing down the y axis, with the x axis being distance along the nanochannel.

FIGS. 7a-7c: Measurements of fabricated features. FIG. 7a) A post structure imaged on AFM, with indicated cross section plotted in FIG. 7b). FIG. 7c) Optical profiler image of microchannel area, indicating the 30 m depth.

FIG. 8: SacI cleaving a YOYO-1-labeled λ-phage DNA confined with 10-nm posts.

### DETAILED DESCRIPTION

A glass-based platform is provided for controlling and visualizing biomolecular (in particular, DNA) interactions in customizable, dynamically adjustable nanoscale (to sub-100-nm) environments (FIG. 1). This platform introduces separate control over a) loading and formatting macromolecular conformations in a nanoscale imaging chamber, and b) exchanging reagent molecules within the chamber via an etched microchannel, and using a sparse array of nanoposts to fix the chamber height. Importantly, this approach decouples sample-loading from reagent-exchange, which facilitates studying multi-step biophysical processes in nanoconfined spaces. The glass-based format is non-absorptive, allows for high spatial resolution of embedded features, and is compatible with scalable wafer-based fabrication, thus translatable to a wide range of applications. This represents a novel advancement in nano-confinement technology, offering a high degree of temporal and physical control over reactions and the ability to time-resolve their evolution and discern their kinetics.

To illustrate, an experiment performed using this nanofluidic platform can proceed via a two-step process: 1) the microfluidic chamber roof is deformed downwards, which squeezes DNA polymers within the open-face, embedded nanotopographies, such as nanogrooves, from the top. This top-loading approach leverages the principle of “Convex Lens-induced Confinement” (CLiC) imaging, which has previously been used to confine molecules for passive, extended observation within a nanoscale slit^{23,24} as well as within nanogrooves approaching the persistence length in cross-sectional dimensions, where deflection off of chamber walls results in high extension.²⁵²⁷ In prior work, the deformed roof was pressed into contact with the floor, creating a curved chamber geometry over the extended imaging region, which resulted in a confinement-gradient and sample depletion. As disclosed herein, a sparse array of posts has been fabricated to establish stable, precise, and homogeneous confinement across the imaging region. 2) With the CLiC lens lowered into place, the microfluidic device enables reagent introduction within the formed nanofluidic imaging region by way of a deep, encircling microchannel, embedded in the chamber floor. Reagents subsequently diffuse from the microchannel into the nanofluidic imaging region through the fixed-height slit.

Disclosed herein is a new, glass-based nanofluidic platform to control and visualize reactions in nanoscale spaces. It leverages the principle of CLiC imaging, which allows for continuously loading and extending biomolecules within customized nanoscale environments. This work introduces temporal control of reagent insertion within a CLiC-nanoslit, subsequent to formatting polymer conformations in embedded nanostructures, by introducing new features, including a microchannel and a nanopost array, into the device design. Because embedded nanotopographies are fabricated on thin coverslips compatible with high-NA oil-immersion objectives, this technology provides real-time visualization of reaction dynamics with down to single-fluorophore sensitivity.

The glass-based design is compatible with scalable, wafer-based fabrication processes, especially once in a miniature format. Of great interest to the biotechnology sector, this platform can be applied to visualize, diagnose and optimize a broad range of enzymatic reactions in real time, such as ligation and labeling reaction steps used in genomic and other diagnostic platforms. In the field of DNA nanotechnology, it can be used to visualize and influence both synthesis and degradation of DNA nanostructures, providing new insights into the structural dependencies of these processes.

Beyond solution-based fluorescence microscopy applications, which provide visualization of dynamics but are limited in spatial resolution, this platform can be used to format and deposit biomolecules on surfaces, which can subsequently be recovered for higher-resolution analysis. For instance, deposition of molecular complexes onto specific surfaces can be chemically initiated after dynamics of the same molecules are observed in solution, which is a subject of current research. Large numbers of deposited complexes, with conformations influenced by the nanotopography, can be analyzed using high-resolution systems such as atomic force microscopy (AFM) or forms of electron microscopy (EM) following device disassembly.

Overall, this nanofluidic platform advances single-molecule confinement and visualization technology by enabling new temporal resolution and control over chemical reaction conditions. This platform can be applied to explore and manipulate a wide range of fascinating biophysical interactions of interest to biological, biotechnological, biophysical, chemical, and materials science research communities, in customized nanoscale environments.

The nanofluidic device includes a flow-cell, formed between flow-cell surfaces. The flow-cell has variable height to provide vertical confinement. The flow-cell can be disassembled for re-use, washing, etc.

The height of the flow-cell is controlled by an external mechanism that moves the flow-cell surfaces relative to one another. At least one flow-cell surface is movable and acted on by the external mechanism. Preferably the top flow-cell surface is movable. The external mechanism is exemplified herein as a Convex Lens-induced Confinement (CLiC)-lens, but other external mechanisms can be used, such as mechanical devices (e.g., a piston, a rod, etc.) or non-mechanical devices (e.g., a source of gas pressure, or a source of fluid pressure).

In some embodiments the flow-cell surfaces are substantially planar, not including any embedded micro- and/or nano-topographies, which are described further below.

In some embodiments, at least one of the flow-cell surfaces (e.g., the top and/or bottom surfaces) includes an array of posts. In some embodiments the top flow-cell surface will include the post array. The heights of the posts in the post array are selected to create a minimum chamber height when the chamber is compressed by the external mechanism such that the posts on one flow-cell surface contact the opposite flow-cell surface. The post array ensures an even confinement around the region of contact of the post array and the flow-cell surfaces, in contrast to prior devices in which deformation of the top surface of a flow cell resulted in a convex area of confinement.

In some embodiments, a post array formed on one or more of the flow-cell surfaces is a hexagonal array of post extrusions. Other geometries of posts can also be used. In instances where posts are formed on both of the top and bottom flow-cell surfaces, the respective arrays each form complementary parts of the whole post array. The spacing of the posts in the array can be determined according to the use of the device, the dimensions of the flow-cell, the height of the posts, the materials used, etc. For example, the posts forming the array can be spaced about 30-μm apart, but other spacings of posts can be used also, such as 10-μm apart, 20-μm apart, 30-μm apart, 40-μm apart, 50-μm apart, 60-μm apart, 70-μm apart, and so on.

The height of the posts in the post array can be selected depending on the particular use of the device, the dimensions of the flow-cell, the spacing of the posts in the array, the materials used, etc. In some embodiments, the posts forming the array are 5-100 nm tall, such as 5, 10, 20, 30, 40, 50, 60, 70, 80, 90 or 100 nm tall. The posts in the array are preferably substantially the same height to provide a uniform distance (minimum chamber height) between the top and bottom surfaces of the flow-cell, e.g., such that the top and bottom surfaces of the flow-cell are substantially parallel. In some embodiments, the post array forms a nanoslit that can be used for introduction and exchange of reagents.

One or more of the flow-cell surfaces includes embedded micro- and/or nano-topographies. Typically the bottom surface (also referred to as the floor herein) contains the embedded micro- and/or nano-topographies. In some embodiments, one of the flow-cell surfaces includes linear embedded nanogrooves. The nanogrooves may be between 10-100 nm deep, 10-100 nm wide, and 100-1000 μm long, though the depth, width and length are only limited by the materials used and the size of the device. In some embodiments, the nanogrooves are about 40-50 nm deep, about 50-nm wide and about 500-μm long. Other micro- and/or nano-topographies can be included instead of nanogrooves, and mixtures of different micro- and/or nano-topographies can be included.

The flow-cell can include a microchannel in the floor of the flow-cell, which can be used for delivery of reagents to the flow-cell. The shape and dimensions of the microchannel can be determined by the size of the device and the particular use of the microchannel. In some embodiments, the microchannel is about 30-rtm-deep and about 200-rpm-wide. In some embodiments, the microchannel can encircle the embedded nano-topographies, such as a nanogroove array, and the imaging region.

The flow-cell surfaces can in some embodiments be made of glass or have glass surfaces, and can in some embodiments be formed by a coverslip. Other materials, such as silicon, also can be used as the material of the flow-cell or flow-cell surfaces. In some instances, it may be preferred to use one material for a portion of the flow-cell, and one or more other materials for one or more other portions of the flow-cell. For example, the portion of the flow-cell that is deformed (e.g., top or “roof”) can be formed of or have a surface of silicon, and other portions of the flow-cell can be formed of or have a surface of glass.

It may be useful or desirable to attach, deposit, or tether one or more biomolecules to a flow-cell surface, such as after the one or more biomolecules is loaded into the flow-cell as described herein, and after pressure is applied on a flow-cell surface such that the flow-cell surface contacts the post array. Subsequently, a reagent can be added to the flow-cell that enables deposition, attachment or tethering of the one or more biomolecules in the flow-cell. Alternatively, a coating on one or more flow-cell surfaces that enables deposition, attachment or tethering of biomolecules can be provided in advance of use. In some embodiments, the reagent or coating that enables deposition, attachment or tethering of biomolecules is a heterobifunctional linker, such as an aminosilane linker. Specific examples of aminosilane linkers include (aminopropyl)triethoxysilane (APTES) and (aminopropyl)dimethylethoxysilane (APDMES).

In addition, the walls of the flow-cell can be coated with a surface-passivation agent, such as polyvinyl pyrrolidone (PVP).

Uses of the nanofluidic devices described herein include methods of analyzing the interaction of biomolecules. Such methods can include loading one or more biomolecules and optionally one or more reagents into the flow-cell of the devices described herein, and applying pressure on a flow-cell surface, such that the flow-cell surfaces contact the post array. The pressure can be applied by any mechanism, but as disclosed herein this is advantageously done by contacting the upper flow-cell surface with a Convex Lens-induced Confinement (CLiC)-lens. The methods also include obtaining images of the one or more biomolecules as further described herein.

### EXAMPLES

### Example 1: Production of Nanofluidic Device

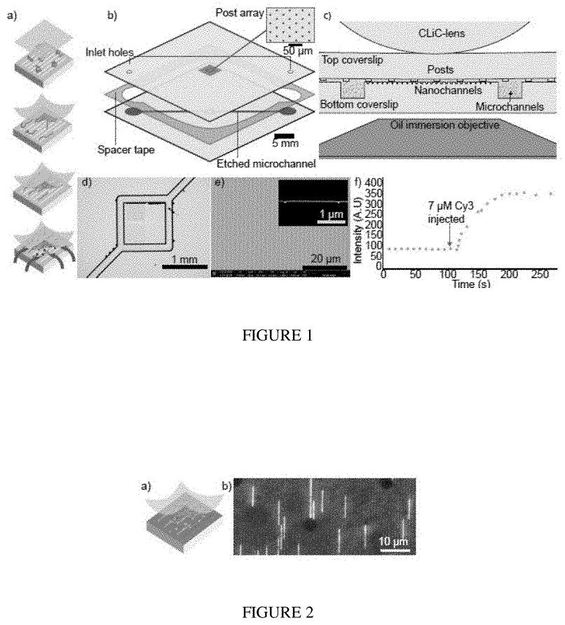

As shown in FIG. 1, the nanofluidic device uses a flow-cell formed between two coverslips which contain embedded micro- and nano-topographies, defined by standard semiconductor fabrication techniques (detailed in Examples 5 and 6). The top flow-cell surface contains a 30-μm-spaced hexagonal array of post extrusions (20-100 nm tall) which, by coming into contact with the bottom surface, creates a nanoslit through which reagents can be exchanged. The bottom chamber surface contains linear embedded nanogrooves, typically 40-50 nm deep, 50-nm wide and 500-μm long, into which DNA polymers can be introduced as the CLiC-lens is lowered (FIG. 2). The total vertical confinement applied to the nanogroove-confined biopolymers, when using 20-nm posts, is approximately 60 to 70 nm, a regime in which loops and folds in the DNA conformation are highly suppressed.²⁸ Further, surface-passivation agents such as polyvinyl pyrrolidone (PVP) used in protein and surfactant experiments will typically coat device walls (with a thickness estimated to be around the hydrodynamic radius, 5.6 nm for 55 kDa PVP29), further reducing the effective confinement experienced by the DNA.

For inserting reagents, a 30-μm-deep, 200-μm-wide microchannel is embedded in the chamber floor, encircling the nanogroove array and imaging region. Since the hydraulic resistance to flowing a fluid reagent is inversely proportional to the cube of the height of a flow channel,³⁰ introducing reagents through the microchannel requires significantly less pressure and hence results in reduced disturbance to the confined DNA. Importantly, this device decouples a) the initial confinement and formatting of the DNA from b) their subsequent interaction with reagent molecules, which facilitates exploration of the effects of confinement, in combination with the serial introduction of reagents, upon DNA. More detail on the experimental procedure is provided in Example 7. To reuse the chamber after an experiment, the CLiC lens is lifted to restore the open chamber geometry, and a wash buffer is delivered through the inlet to rinse the glass chamber. The device can be recovered by dissolving the adhesive in an acetone bath, after which the cleaning process can begin again. The figures delineate results from a single experiment's single field of view, for each reagent introduced, illustrating ease of use and potential for high throughput.

### Example 2: Dynamic Response of YOYO-1-DNA to Changes in Ionic Strength

The dynamic effect of increasing the ionic strength of solution on DNA-YOYO-1 complexes confined within nanogrooves were visualized, and outstanding questions on how YOYO-1 influences DNA length and stiffness were addressed. The nature of YOYO-1 binding to DNA molecules remains of keen interest to the nanofluidics field, with differing viewpoints among researchers,³¹⁻³⁷ especially given its frequent use as a stain.

It is generally understood that fluorescing YOYO-1 dye is electrostatically coupled to the negatively charged backbone of DNA, and its bis-intercalating fluorescent ring structures interleave between the -stacked bases. More interestingly, recent experiments have suggested a complex kinetic picture. Multiple modalities of YOYO-DNA binding have been proposed, ranging from “groove-binding” to “intercalating” modes, with different strengths of association.^{33,38}

Because YOYO-1 dye molecules are thought to interleave within the DNA backbone in the “intercalating mode”, one may expect YOYO-1-DNA intercalation to contribute to an increase in persistence length. However, reported measurements are divided on the effect of YOYO-1 on persistence length.³¹ While some experiments report an increase in persistence length proportional to overall increase in contour length as a function of YOYO-1 addition,^{39,40} other experiments suggest that the persistence length remains unchanged or decreases with YOYO-1.^{31,37}

As the association of YOYO-1 dye with DNA is mediated by interaction of four positive charges on the dye with the negatively charged DNA backbone, it is possible to probe this interaction by adjusting the ionic strength. Prior measurements of length have been performed in side-loaded nano-slits and nanochannels, following incubation in buffer solutions of desired ionic strengths.⁴⁰⁻⁴² This approach obscures dynamic information which can be made available by visualizing molecules during the buffer exchange.

Additionally, prior work has not directly quantified the dynamic change in DNA intensity which is reported herein. Dynamic imaging during solution exchange offers the ability to directly correlate the change in YOYO-1 fluorescence, and hence YOYO-1 binding with DNA, with the change in DNA length. Integrated intensity along the molecules is expected to scale linearly with the number of bound YOYO-1 at a ratio below approximately one dye per 10 basepairs,⁴³ and dye molecules that unbind from DNA emit over 1000 times less fluorescence.

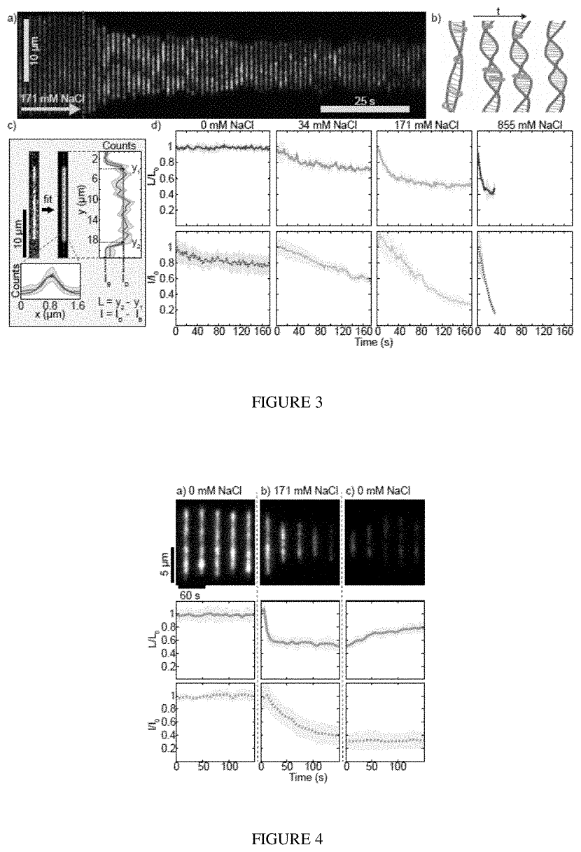

The capabilities of the disclosed nanofluidic platform were leveraged to measure dynamic changes in length and integrated intensity of single DNA molecules introduced to buffers of increasing ionic strength, and to distinguish between two qualitatively different, salt-induced DNA dynamics, which occur on different timescales: 1) ionic screening of the polymer and 2) de-intercalation of YOYO-1 from the polymer backbone (FIGS. 3 a, 3b). Specifically, time-resolved measurements of DNA length and integrated intensity as a function of ionic strength of solution were performed. The equilibration time was expected to be bounded within 100 s as the Na⁺ and Cl⁻ ions are smaller than the fluorescent dye molecules shown in FIG. 1. FIG. 3c delineates fluorescent images of DNA molecules during buffer exchange, which were fit for the DNA extension L and integrated intensity I as functions of time, using procedures established in Berard et al.²⁶ To simplify, quantitative results are shown for DNA molecules which do not overlap with other molecules, do not photocleave during imaging and do not escape the field of view. DNA length was observed to decrease when a higher-ionic-strength buffer was introduced and to stabilize to a final value within 80 s (FIG. 3d), commensurate with diffusion times for these molecules over a few hundred microns from the microchannel. This fractional length change is in agreement with other published results⁴⁰ but the time-resolved behaviour was not previously observed.

The DNA length was observed to reach a final value well before the YOYO-1 binding equilibrated, as indicated by the DNA brightness continuing to change. The data suggests that YOYO-1 molecules take longer to dissociate from the DNA backbone, potentially due to complex, multi-step unbinding modalities, compared to the timescale for the polymer length to reduce due to increased ionic concentration.

That there was a latter period wherein YOYO-1 brightness decreased while the length didn't change indicates that there were fluorescing modes of binding that do not contribute to DNA mechanical properties, including persistence length. One potential explanation is that during the unbinding process, the YOYO-1 dye transitions between the proposed “intercalating” vs. “groove-binding” modes, but continues to exhibit high fluorescence in “groove-binding” modes without contributing the contour length.

Additional experiments were performed to demonstrate reversibility and to control for the potential impact of photobleaching during microscopy experiments. Using a longer laser shutter time (300 ms between frames, during which the molecules are not illuminated) photobleaching effects were reduced to negligible levels over the time frame of the experiment (FIG. 4a). Buffer containing 171 mM NaCl was introduced (FIG. 4b) and then replaced with buffer containing no NaCl (FIG. 4c). Suppressing photobleaching clarifies the contrast in timescales over which length and intensity vary, which, respectively, are from less than 50 s to over 150 s.

FIG. 4 delineates recovery of extension but not intensity, consistent with the release of the dye. The example molecule exhibited an initial extension with no NaCl of 9.02 m, with a standard deviation of 0.38 m over the imaging period. The extension recovered only to about 80% of its initial value, for a few potential reasons. The non-recovered extension can be due to depletion of dye molecules, or to residual salt in the chamber, after the second buffer exchange. In quantitatively assessing these options, the established measurement of the contour-length contribution of intercalated YOYO-1 dye were used, reported as 0.5 nm per YOYO-1 molecule.³¹ The staining ratio of 1 dye molecule per 10 bp (natively 0.33 nm per base pair) corresponds to a 15% increase in contour length. If it is taken from the fluorescence measurement that the DNA lost between 60 and 70% of its bound dye, then the lost dye could only account for 10% of the extension. The other 10% of unrecovered extension indicates some other effect on DNA nanochannel extension. One possibility is that NaCl has been shown to decrease the negative surface charge in glass nanochannels⁴⁴ which in turn has been shown to affect the extension of nanoconfined DNA.⁴⁵ Such surface charge modification can account for lower polymer confinement in this application; this can be further suppressed by varying surface-passivation agents. In summary, two dynamic effects of ionic strength on YOYO-1-complexed DNA have been successfully demonstrated, probing open questions on the influence of salt and intercalating dyes upon DNA properties.

### Example 3: DNA Compaction with the Cationic Surfactant CTAC

Further to this exploration, the disclosed technology facilitates study of DNA compaction in a wide range of contexts, as well as study of reactions with enzymes and proteins sensitive to DNA topology. Control of DNA compaction has important consequences in DNA packing, replication and transcription. The human genome, for example, would correspond to two meters (end-to-end) of naked native DNA, but is stored in a cell nucleus about 10 microns in diameter, representing a reduction in scale by several orders of magnitude. There is much interest in how this transition comes about, and many methods have been applied to recreate and visualize DNA compaction in vitro with controlled, few component systems.⁴⁶

Previous work has revealed DNA compaction processes in specific confined and crowded environments, for instance environments in which the DNA is initially stretched electrophoretically.²¹ Long-time compaction dynamics of DNA with a nucleoid-structuring protein have also been observed.²² It has been hypothesized that despite a variety of approaches to achieving DNA condensation—surfactants, divalent salts, molecular crowding—the condensed conformation is “universal”, a tightly coiled toroid.⁴⁷

Here, dynamic, temporally controlled compaction of DNA polymers immersed in a cationic surfactant solution were visualized. For these studies, the surfactant cetyl trimethyl ammonium chloride (CTAC) was used, which has a positive head group and a long hydrophobic tail. The positive charges are hypothesized to bind along the length of the DNA and cause the DNA to condense and eventually precipitate.⁴⁸ The dynamics of this condensation transition were explored with single-molecule resolution to establish a transition timescale.

To suppress interactions with the walls of the nano-channels, which require more care in these experiments to minimize non-specific adsorption, the glass was treated with the fluorinated silane 1H,1H,2H,2H-perfluorooctyldimethylchlorsilane (FOCS). This created a hydrophobic, inert surface-layer, complemented by an in-situ coating with polyvinyl pyrrolidone (PVP). To accommodate the additional confinement due to the inert polymer layer, 40-nm posts were used. CTAC and YOYO-1 both associate to DNA via ionic interactions; to avoid the effect of this competition in these experiments, the λ-phage DNA was labeled covalently with Cy3 moieties in these experiments (which do not increase DNA extension, as intercalating dyes such as YOYO-1 do), resulting in shorter molecules compared to images which use YOYO-1 labeling. Photobleaching was suppressed by an enzymatic oxygen scavenging system. The compaction buffer contained 92 mM CTAC.

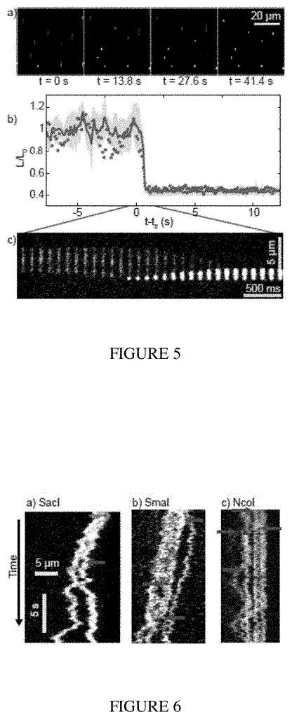

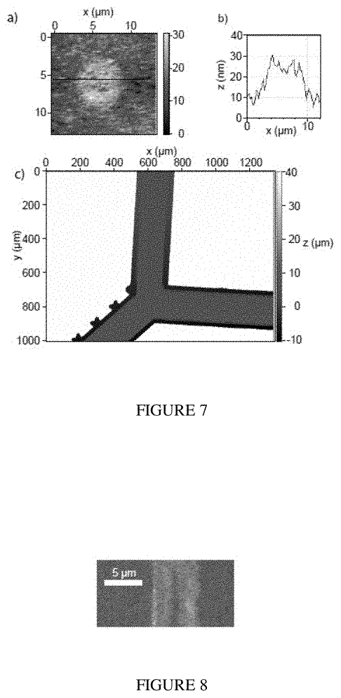

After introduction of the CTAC buffer, a compaction transition in exposed DNA molecules was visualized. The reaction progressed within minutes toward the center of the chamber from the microchannel area, representing the diffusing front of the CTAC solution (FIG. 5). As the CTAC solution approached the DNA polymers, the DNA was visualized being pulled toward the solution interface, and for compaction to proceed along the polymer length. At the end of the time-course, the polymer finally appeared adsorbed onto the chamber walls. The “compacted state” was empirically characterized as a 2.5-fold increase in local intensity as well as decrease in polymer length to 40% of its initial value. A consistent feature among measurements of many molecules, the compaction nucleated at the end of the DNA molecule closest to the microchannel and proceeded along the length of the polymer in 1.5 s. Eventually, the compacted molecules adsorbed onto the surface and did not exhibit resolvable fluctuations in intensity or length (FIG. 5b,c). In the compacted state, the DNA were still confined by the nanogroove and top surfaces: rather than compacting into a spherical globule, as was observed in unconfined experiments, a collapse into characteristic “chain of smaller globules” aligned with the highly confined nanogroove was observed. It cannot be ruled out that this could be due to polymers adsorbing before compaction is complete, and the resultant conformation may be incorporating an effect of the high density PVP layer on the surface. However, the 600-nm size of chained globules was consistent among observed molecules. Quantitatively, it is interesting to point out that this in situ compaction occurred on a larger length scale than the λ-phage virus packing its DNA in its 60-nm capsid.⁴⁹ These results motivates and facilitates further study of a wide range DNA compaction processes as a function of nanoscale confinement, surfactant tail length, and reagent conditions which have otherwise remained out of reach.

### Example 4: DNA Cleavage Via Restriction Enzymes

Next, a third nanofluidic application corresponding to the function of restriction enzymes in nanoscale environments was explored. Restriction enzymes are routinely used to cut DNA at specific sites, with a wide range of applications in biochemical processes.⁵⁰ Previous work has demonstrated the use of restriction enzymes in optically mapping genetic material fixed to agarose gels or glass,⁵¹ as well as in nanochannels.⁵² Restriction mapping offers a way to determine long-range structure in genetic material (over many kbp), highlighting, for example, large transpositions which may be difficult to observe in state-of-the-art basepair-level sequencing techniques.⁵³ Restriction mapping in nanochannels has the benefit of collecting sufficient mapping information from a single molecule, as the ensemble-average of conformational fluctuations can be determined from a video of a diffusing polymer. In contrast, samples fixed to a surface require averaging over many molecules,⁵¹′5² as each molecule is in one conformation.

The disclosed technology was applied to study temporally controlled single molecule restriction mapping. The separation of steps between 1.) extending DNA and 2.) introducing the activating reagent, was achieved by first lowering the CLiC-lens to confine DNA in the nanogrooves and subsequently introducing magnesium ions through the microchannel. Unlike previous technology, the device does not require use of electrodes to electrophoretically load DNA or magnesium ions.⁵² Restriction of DNA molecules that are confined in nano-geometries smaller than previously reported was demonstrated, using 100, 40, and 5-nm posts.

Using this approach, reactions of more than 30 molecules in one field of view can be visualized. As a demonstration of use of this method at high levels of confinement, direct visualization of enzymatic activity was performed using 3 different post heights and 3 restriction enzymes acting on λ-phage DNA: SacI (producing two four-base 3′ overhangs at 24772 and 25877), SmaI (three blunt ends at 19399, 31619, and 39890), and NcoI (four four-base 5′ overhangs at 19329, 23901, 27868, and 44248). For SacI, SmaI, NcoI 1, 3, and 4 cuts were resolved (labeled by red arrows in FIG. 6) corresponding to fragments larger than about 1 kbp, respectively. As in prior work,⁵² cuts associated with smaller fragments are not shown.

SacI, SmaI, and NcoI reactions were visualized using chambers with 100, 40 and 5-nm posts, respectively. Data for SacI with 10-nm posts is shown in FIG. 8. The chamber was pre-passivated with a 10% 55 kDa PVP solution, as magnesium is a divalent cation that can cause DNA to adsorb to glass. All restriction experiment buffers contained 20 mM Tris-HCl (pH 8), 50 mM potassium chloride, 1 mM dithiothreitol (DTT), 10% 55 kDa PVP and 3% BME. DNA was immersed in buffer with restriction enzymes (NEB) at concentrations of 2,000 units/mL for SacI and SmaI and 1,000 units/mL for NcoI, where a unit is defined as the amount of enzyme required to digest 1 g of λ-phage DNA in 1 hr at optimal temperature (23° C. for SmaI, 37° C. for SacI and NcoI).

Experiments performed in the absence of magnesium or enzyme did not show cleavage during the duration of the experiment, which was over 5 minutes. After introduction of buffer containing 1 mM MgCl₂, most cleavage events occurred within two minutes. As expected, experiments with SacI, SmaI, and NcoI exhibited 1, 3 and 4 cuts with resolvable fragments, respectively (FIG. 6)], even with the extension decreased by the high-salt incubation and the introduction of MgCl₂. The results demonstrate the functionality of a suite of enzymes in the presented nanoconfined environments. Restriction enzyme activity was seen with posts as small as 5 nm and no discernable effect of post height was observed on activity for the presented experimental conditions. Nanochannel restriction mapping has been previously demonstrated to compare favorably to pre-existing optical mapping methods, e.g. by strongly decreasing the amount of sample required, and by regulating the conformations and fluctuations of DNA molecules along the nanochannel.⁵² The disclosed device can contribute to further development by allowing a wide range of in-situ modifications to restriction processes.

### Example 5: Device Fabrication

Nanogroove arrays were fabricated as previously published.²⁷ Piranha-cleaned, 170-μm-thick glass wafers (Schott, D263) were patterned with gold alignment marks by standard UV lithography and a lift-off process. After a plasma clean, the coverslips were spin-coated with ZEP520A, a positive electron-beam resist, with a thermally-evaporated aluminum discharge layer on top of that. The ZEP resist is exposed using electron-beam lithography (VB6 UHR EWF; Vistec Lithography) to define nanochannel arrays, with 500-micron X 50-nanometer nanochannels, spaced by 1-8 microns. The exposed pattern was then developed and etched by RIE for depths of about 50 nanometers. The wafers were then diced into 9 25-mm-square devices.

Microchannels were etched on clean coverslips that were previously patterned with nanochannel arrays, surrounding this central 1.2-mm-square region with a 200-micron-wide, 30-micron-deep microchannel. Sequentially sputtered chromium and gold was etched using UV lithography of a positive photoresist followed by metal etchants, to create an etch mask for hydrofluoric acid (HF). HF is highly corrosive and toxic, and should be used only with full acid-resistant equipment, complete body coverage, using goggles and a face mask, and only in a designated area. Immersion in an HF solution subsequently etched the exposed glass to a depth of 30 microns. The channel was designed to run between two corners of the coverslip. To enable sample insertion and removal, the entry ends to the channel were positioned below 1-millimeter holes in the top coverslip.

Standard microscopy coverslips were used to create the chamber roof, into which inlet holes were sandblasted at the corners. The piranha-cleaned sandblasted coverslips were subsequently patterned with a sparse hexagonal array of 10-μm-wide circles in positive UV-sensitive resist using UV lithography. The glass was next placed in an RIE chamber for etching to a depth of 20 to 100 nm, leaving the hexagonal array as extrusions on the surface.

Each coverslip underwent a rigorous cleaning procedure before use. Because the flow cells were recovered and re-used between experiments, it was critical for residues to be removed. Coverslips were treated in sequential 1% Hellmanex at 50° C., acetone at 50° C., isopropanol at 50° C., and finally 2M KOH in ethanol to remove macroscopic residues, which was followed by a clean in 3:1 sulfuric acid to 30% hydrogen peroxide piranha solution. This approach thoroughly removed any organic contaminants from the surface and exposed hydroxyl groups. Coverslips were rinsed thoroughly with DI water between all steps. Coverslips were then soaked in 100 mM KOH, rinsed with DI water, and were ready for use or for further treatments. Since the CLiC microscopy experiments presented herein require nanoscale surface contact, rigorous cleaning is essential.

### Example 6: Device and Sample Preparation

For experiments requiring a particularly inert surface, coverslips were next treated with 1H,1H,2H,2H-perfluorooctyldimethylchlorosilane (FOCS), purchased from Alfa Aesar, prior to use. Coverslips were placed in a vacuum desiccator for one hour with 5-10 μL of FOCS for vapor-phase deposition. The coating could be assessed by examining the shape of a water droplet on the coverslip.

A successful coating corresponds to a highly hydrophobic surface. For most experiments, λ-phage DNA (48.5 kbp, New England Biosciences) was stained in YOYO-1 dye (Life Technologies) to achieve a mixture in solution of 1 dye molecule for every 10 basepairs (16.6 μM for 100 ng/L DNA). All solutions were diluted in 0.5×TBE (45 mM tris borate, ImM EDTA; pH 7.8). The mixture was incubated for an hour at 55° C. for uniform dye distribution. For some experiments, λ-phage DNA was stained with Cy3 fluorescent dye using a non-enzymatic labeling kit (Mirus). The stained DNA was stored at 4° C. at a concentration of 100 ng/μL.

Immediately prior to a microscopy experiment, fluorescently-labeled DNA was diluted from 100 to 50 ng/L in 0.5×TBE with 3% β-mercaptoethanol (BME) by volume, for YOYO-1-labeled DNA, or 384 μg/mL protocatechuic acid (PCA) and 34 μg/mL protocatechuate-3,4-dioxygenase (PCD), for Cy3-labeled DNA.

### Example 7: Experimental Procedure for Device Use

A flow-cell assembled with 10-rpm double-sided adhesive (Nitto Denko) was placed on a sample plate above the inverted fluorescence microscope (Nikon Ti-E), and sealed to a microfluidic chuck with a rubber gasket and thumbscrews.²⁴ The chuck allowed sample loading by applying air pressure at the inlet and outlet holes. The tube holding the CLiC-lens was mounted above the chuck and was lowered by a picomotor (Newport 8392), for coarse control, and piezo actuator (Physik Instrumente P-602.1SL), for fine control, after the flow cell was positioned underneath the lens by a custom translation stage controlled by micrometers. The CLiC device and translation stage were positioned relative to an oil immersion objective (CFI Apo TIRF 100×) by an additional translation stage (Physik Instrumente P-545) controlled by a joystick.

To prevent surface adsorption during some experiments, the chamber was washed with 30 μL of 10% 55-kDa polyvinylpyrrolidone (PVP) containing the protocatechuic acid (PCA)/protocatechuate-3,4-dioxygenase (PCD) oxygen scavenging system, and then incubated for 15 minutes before rinsing with 60 μL of 1% 55-kDa PVP.²⁴

The diluted, fluorescently-labeled DNA was loaded into the chamber and the CLiC-lens was lowered by the piezo device until contact was made between the posts and the bottom coverslip, indicated by imaging the interference pattern of the laser excitation. Glass, once in contact, became characteristically dark and remained dark with further lowering of the CLiC-lens.

After contact was made, the CLiC-lens was typically lowered an additional 10-15 m to further deform the glass and ensure contact across an extended area²⁴, as well as to provide stability under pressure during sample-loading. Both glass surfaces deform under the physical pressure, but the post array ensures an even confinement around the region of contact. Using a 488-nm laser for YOYO-1 excitation or 532-nm laser for Cy3 excitation, and standard emission filters for these fluorophores' spectra, the DNA extension in nanogrooves was then observed using an EM-CCD camera (Andor iXon Ultra). For different experiments, DNA molecules extended in the nanochannels to 60-90% of their contour length, depending on solution conditions and the applied confinement. Sub-100-nm confinement yielded up to 90% polymer extension, corresponding to the Odijk regime of confinement defined by deflection of the polymer off the nanochannel walls.

Excess solution was removed from the chuck inlet chamber prior to pipetting in subsequent reagents. Reagents were typically diluted in 0.5×TBE with the appropriate antioxidation reagents (BME or PCA/PCD) to suppress photobleaching. After inserting reagents in the chuck chamber, negative pressure was applied to the chamber via the outlet to load reagent into the microchannels.

After an experiment, reagents can be rinsed from the chamber by lifting the CLiC lens and flowing large amounts of a wash buffer through the chamber. To recover the flow cell, the adhesive is dissolved in acetone overnight, after which it can undergo the cleaning process for a new experiment.

### REFERENCES

(1) Sintra, T. E.; Ventura, S. P.; Coutinho, J. A. J. Mol. Catal. B: Enzym. 2014, 107, 140-151.
(2) Deniz, A. A.; Mukhopadhyay, S.; Lemke, E. A. J. R. Soc. Interface 2008, 5, 15-45.
(3) Reisner, W.; Pedersen, J. N.; Austin, R. H. Rep. Prog. Phys. 2012, 75, 106601.
(4) Thompson, J. F.; Milos, P. M. Genome Biol. 2011, 12, 1-10.
(5) Metzker, M. L. Nat. Rev. Genet. 2009, 11, 31-46.
(6) Küchler, A.; Yoshimoto, M.; Luginbuihl, S.; Mavelli, F.; Walde, P. Nat. Nanotechnol. 2016, 11, 409-420.
(7) Levy, S. L.; Craighead, H. G. Chem. Soc. Rev. 2010, 39, 1133-1152.
(8) Persson, F.; Tegenfeldt, J. O. Chem. Soc. Rev. 2010, 39, 985-999.
(9) Joo, C.; Balci, H.; Ishitsuka, Y.; Buranachai, C.; Ha, T. Annu. Rev. Biochem. 2008, 77, 51-76.
(10) Neely, R. K.; Deen, J.; Hofkens, J. Biopolymers 2011, 95, 298-311.
(11) Lam, E. T.; Hastie, A.; Lin, C.; Ehrlich, D.; Das, S. K.; Austin, M. D.; Deshpande, P.; Cao, H.; Nagarajan, N.; Xiao, M.; Kwok, P.-Y. Nat. Biotechnol. 2012, 30, 771-776.
(12) Xiao, M.; Phong, A.; Ha, C.; Chan, T.-F.; Cai, D.; Leung, L.; Wan, E.; Kistler, A. L.; DeRisi, J. L.; Selvin, P. R.; Kwok, P.-Y. Nucleic Acids Res. 2007, 35, e16.
(13) Zohar, H.; Muller, S. J. Nanoscale 2011, 3, 3027-3039.
(14) Cai, W.; Aburatani, H.; Stanton, V. P.; Housman, D. E.; Wang, Y. K.; Schwartz, D. C. Proc. Natl. Acad. Sci. U.S.A. 1995, 92, 5164-5168.
(15) Tegenfeldt, J. O.; Prinz, C.; Cao, H.; Chou, S.; Reisner, W. W.; Riehn, R.; Wang, Y. M.; Cox, E. C.; Sturm, J. C.; Silberzan, P.; Austin, R. H. Proc. Natl. Acad. Sci. USA 2004, 101, 10979-10983.
(16) Freitag, C.; Noble, C.; Fritzsche, J.; Persson, F.; Reiter-Schad, M.; Nilsson, A. N.; Granéli, A.; Ambjörnsson, T.; Mir, K. U.; Tegenfeldt, J. O. Biomicrofluidics 2015, 9.
(17) Dorfman, K. D.; King, S. B.; Olson, D. W.; Thomas, J. D. P.; Tree, D. R. Chem. Rev. 2013, 113, 2584-2667, PMID: 23140825.
(18) Baldessari, F.; Santiago, J. G. J Nanobiotechnology 2006, 4, 1-6.
(19) Alrifaiy, A.; Lindahl, O. A.; Ramser, K. Polymers 2012, 4, 1349-1398.
(20) Zhang, C.; Jiang, K.; Liu, F.; Doyle, P. S.; van Kan, J. A.; van der Maarel, J. R. C. Lab Chip 2013, 13, 2821-2826.
(21) Zhang, C.; Shao, P. G.; van Kan, J. A.; van der Maarel, J. R. C. Proc. Natl. Acad. Sci. U.S.A. 2009, 106, 16651-16656.
(22) Zhang, C.; Guttula, D.; Liu, F.; Malar, P. P.; Ng, S. Y.; Dai, L.; Doyle, P. S.; van Kan, J. A.; van der Maarel, J. R. C. Soft Matter 2013, 9, 9593-9601.
(23) Leslie, S. R.; Fields, A. P.; Cohen, A. E. Anal. Chem. 2010, 82, 6224-6229, PMID: 20557026.
(24) Berard, D.; McFaul, C. M. J.; Leith, J. S.; Arsenault, A. K. J.; Michaud, F.; Leslie, S. R. Rev. Sci. Instrum. 2013, 84, 103704.
(25) Odijk, T. Macromolecules 1983, 16, 1340-1344.
(26) Berard, D. J.; Michaud, F.; Mahshid, S.; Ahamed, M. J.; McFaul, C. M. J.; Leith, J. S.; Bérubé, P.; Sladek, R.; Reisner, W.; Leslie, S. R. Proc. Natl. Acad. Sci. USA 2014, 111, 13295-300.
(27) Berard, D. J.; Shayegan, M.; Michaud, F.; Henkin, G.; Scott, S.; Leslie, S. Appl. Phys. Lett. 2016, 109, 033702.
(28) Tree, D. R.; Wang, Y.; Dorfman, K. D. Phys. Rev. Lett. 2013, 110, 208103.
(29) Armstrong, J.; Wenby, R.; Meiselman, H.; Fisher, T. Biophys. J. 2004, 87, 4259-4270.
(30) Lee, H.; Ham, D.; Westervelt, R. CMOS Biotechnology; Springer: USA, 2007.
(31) Kundukad, B.; Yan, J.; Doyle, P. S. Soft Matter 2014, 10, 9721-9728.
(32) Sischka, A.; Toensing, K.; Eckel, R.; Wilking, S. D.; Sewald, N.; Ros, R.; Anselmetti, D. Biophys. J. 2005, 88, 404-411.
(33) Reuter, M.; Dryden, D. T. Biochem. Biophys. Res. Commun. 2010, 403, 225-229.
(34) Günther, K.; Mertig, M.; Seidel, R. Nucleic Acids Res. 2010, 38, 6526-6532.
(35) Rye, H. S.; Yue, S.; Wemmer, D. E.; Quesada, M. A.; Haugland, R. P.; Mathies, R. A.; Glazer, A. N. Nucleic Acids Res. 1992, 20, 2803-2812.
(36) Rye, H.; Dabora, J.; Quesada, M.; Mathies, R.; Glazer, A. Anal. Biochem. 1993, 208, 144-150.
(37) Maaloum, M.; Muller, P.; Harlepp, S. Soft Matter 2013, 9, 11233-11240.
(38) Paik, D. H.; Perkins, T. T. Angew. Chem. Int. Ed. 2012, 51, 1811-1815.
(39) Chu, S.; Quake, S. R.; Babcock, H. Nature 1997, 388, 151-154.
(40) Reisner, W.; Beech, J. P.; Larsen, N. B.; Flyvbjerg, H.; Kristensen, A.; Tegenfeldt, J. O. Phys. Rev. Lett. 2007, 99, 058302.
(41) Hsieh, C.-C.; Balducci, A.; Doyle, P. S. Nano Lett. 2008, 8, 1683-1688, PMID: 18459741.
(42) Zhang, C.; Zhang, F.; van Kan, J. A.; van der Maarel, J. R. C. J. Chem. Phys 2008, 128.
(43) Biebricher, A. S.; Heller, I.; Roijmans, R. F. H.; Hoekstra, T. P.; Peterman, E. J. G.; Wuite, G. J. L. Nat. Commun. 2015, 6, 7304.
(44) Raider, S. I.; Gregor, L. V.; Flitsch, R. J. Electrochem. Soc 1973, 120, 425-431.
(45) Uba, F. I.; Pullagurla, S. R.; Sirasunthorn, N.; Wu, J.; Park, S.; Chantiwas, R.; Cho, Y.-K.; Shin, H.; Soper, S. A. Analyst 2015, 140, 113-126.
(46) Bloomfield, V. A. Curr. Opin. Struct. Biol. 1996, 6, 334-341.
(47) Hud, N. V.; Vilfan, I. D. Annu. Rev. Biophys. Biomol. Struct. 2005, 34, 295-318, PMID: 15869392.
(48) Mel'nikov, S. M.; Sergeyev, V. G.; Yoshikawa, K. J. Am. Chem. Soc. 1995, 117, 2401-2408.
(49) Bayer, M. E.; Bocharov, A. F. Virology 1973, 54, 465-475.
(50) Pingoud, A.; Jeltsch, A. Nucleic Acids Res. 2001, 29, 3705-3727.
(51) Schwartz, D.; Li, X.; Hernandez, L.; Ramnarain, S.; Huff, E.; Wang, Y. Science 1993, 262, 110-114.
(52) Riehn, R.; Lu, M.; Wang, Y.-M.; Lim, S. F.; Cox, E. C.; Austin, R. H. Proc. Natl. Acad. Sci. U.S.A. 2005, 102, 10012-10016.
(53) Goodwin, S.; McPherson, J. D.; McCombie, W. R. Nat. Rev. Genet. 2016, 17, 333-351.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. In addition, any combination of two or more of such features, systems, articles, materials, kits, and/or methods, if such features, systems, articles, materials, kits, and/or methods are not mutually inconsistent, is included within the inventive scope of the present disclosure.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or,” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited. All references, patents and patent applications disclosed herein are incorporated by reference with respect to the subject matter for which each is cited, which in some cases may encompass the entirety of the document.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A nanofluidic device comprising
a flow-cell formed between flow-cell surfaces, wherein at least one of the flow-cell surfaces comprises an array of posts, and wherein the flow-cell has variable height.

2. The nanofluidic device of claim 1, wherein the post array ensures an even confinement around the region of contact of the post array and the flow-cell surfaces, wherein the contact of the post array and the flow-cell surfaces creates a minimum chamber height.

3. The nanofluidic device of claim 1 or claim 2, wherein one or more of the flow-cell surfaces comprise embedded micro- and/or nano-topographies.

4. The nanofluidic device of any one of claims 1-3, wherein one or more of the flow-cell surfaces are formed by a coverslip.

5. The nanofluidic device of any one of claims 1-4, wherein one or more of the flow-cell surfaces comprise glass and/or silicon surfaces or are formed of glass and/or silicon.

6. The nanofluidic device of any one of claims 1-5, wherein one or more of the flow-cell surfaces comprises a hexagonal array of post extrusions.

7. The nanofluidic device of any one of claims 1-6, wherein the posts forming the array are 30-μm-spaced posts.

8. The nanofluidic device of any one of claims 1-7, wherein the posts forming the array are 5-100 nm tall.

9. The nanofluidic device of any one of claims 1-8, wherein the post array forms a nanoslit.

10. The nanofluidic device of any one of claims 1-9, wherein one of the flow-cell surfaces comprises linear embedded nanogrooves.

11. The nanofluidic device of claim 10, wherein the nanogrooves are about 40-50 nm deep, about 50-nm wide and about 500-μm long.

12. The nanofluidic device of any one of claims 1-11, wherein walls of the flow-cell are coated with a surface-passivation agent, optionally polyvinyl pyrrolidone (PVP) and/or a heterobifunctional linker, optionally (aminopropyl)triethoxysilane (APTES).

13. The nanofluidic device of any one of claims 1-12, wherein the flow-cell comprises a floor, and the floor of the flow-cell comprises a microchannel.

14. The nanofluidic device of claim 13, wherein the microchannel is about 30-μm-deep and about 200-μm-wide.

15. The nanofluidic device of any one of claims 1-14, wherein the microchannel encircles the nanogroove array and imaging region.

16. The nanofluidic device of any one of claims 1-15, wherein the variable height of the flow-cell is controlled by an external mechanism.

17. The nanofluidic device of any one of claims 1-16, wherein the flow-cell surfaces are substantially planar.

18. The nanofluidic device of any one of claims 1-17, wherein the flow-cell can be disassembled.

19. A method for analyzing the interaction of biomolecules, comprising
loading one or more biomolecules and optionally one or more reagents into the flow-cell of any one of claims 1-18, and
applying pressure on a flow-cell surface, such that the flow-cell surface contacts the post array.

20. The method of claim 19, wherein the pressure is applied by contacting the upper flow-cell surface with a Convex Lens-induced Confinement (CLiC)-lens.

21. The method of claim 19 or claim 20, further comprising obtaining images of the one or more biomolecules.

22. The method of any one of claims 19-21, further comprising adding a reagent or coating to the flow-cell that enables deposition, attachment or tethering of biomolecules to one or more surfaces of the flow-cell.

23. The method of claim 22, wherein the reagent or coating is (aminopropyl)triethoxysilane (APTES).
